(19) United States
(12) Patent Application Publication
(10) Pub. No.: US 2018/0168795 A1
(43) Pub. Date: Jun. 21, 2018

(54) VALVED CONDUIT AND METHOD FOR FABRICATING SAME

(71) Applicant: PECA Labs, Inc., Etna, PA (US)

(72) Inventors: C. Douglas BERNSTEIN, Pittsburgh, PA (US); Arush KALRA, Pittsburgh, PA (US)

(21) Appl. No.: 15/737,186

(22) PCT Filed: Jun. 20, 2016
(86) PCT No.: PCT/US16/38302

§ 371 Date: Dec. 15, 2017

Related U.S. Application Data
(60) Provisional application No. 62/181,521, filed on Jun. 18, 2015.

(51) Int. Cl. A61F 2/06; A61F 2/24

(52) U.S. Cl. CPC A61F 2/06; A61F 2/2415; A61F 2/2412; A61F 2240/001; A61F 2220/0058; A61F 2220/0075; A61F 2002/068; A61F 2220/005

(57) ABSTRACT

Valved conduits having leaflet structures that do not contact an inner surface of a conduit in which they are formed are described herein.

### CROSS REFERENCE TO RELATED APPLICATION

This application claims priority from U.S. Provisional No. 62/181,521, entitled “Valved Conduit and Method for Fabricating Same,” filed Jun. 18, 2015, the entirety of which is hereby incorporated by reference.

### BACKGROUND

In attempts to provide valved conduits to patients undergoing, for example, the Norwood Procedure, some institutions have created hybrid valved conduits by attaching a homograft valve and to a synthetic conduit. While this approach may offer some benefit over a valve-less conduit, homograft valves impart a number of other problems, including calcification, immune rejection, and occasional perforation of valve leaflets, among others. ePTFE valved conduits have been shown to greatly resist these issues, which has led to improved results compared to homograft for similar pediatric cardiac surgical procedures where larger-diameter valved conduits are used. Following this precedent, a properly-functioning valved small-diameter RV-PA conduit would likely maintain the improved short-term results of the valveless RV-PA conduits while improving mid-term and long-term results as well. The techniques and design principles used to create such a device may provide utility for additional applications as well, such as valved conduits or valved stents for other anatomical positions or other pathologies.

### BRIEF SUMMARY OF THE INVENTION

Various embodiments are directed to valves including: a conduit having an inner conduit surface and an outer conduit surface; and a valve structure comprising one or more leaflet, each leaflet having an outer sinus edge, an inner sinus edge, an open sinus edge and a fan, attached to the inner surface of the conduit at the outer sinus edge and inner sinus edge, wherein the open sinus edge is suspended below the inner surface of the conduit by a depth creating a sinus between the leaflet and the inner conduit surface. In some embodiments, each leaflet has a substantially triangular shape. In certain embodiments, each open sinus edge has a width that is less than the circumference of the conduit between fixture points of the leaflet to the conduit, and in particular embodiments, the outer sinus edge and the inner sinus edge may be attached to the conduit by a fluid impervious connection selected from the group consisting of suturing, welding, fusion, applying an adhesive, and combinations thereof. In various embodiments, the conduit and valve structure may each individually be composed of any biocompatible and hemocompatible polymer, and in such embodiments, the biocompatible and hemocompatible polymer may be a fluoropolymer such as, but not limited to, polytetrafluoroethylene, expanded polytetrafluoroethelyne, polyester, polyethylene terephthalate, polydimethylsiloxane, polyurethane, and combinations thereof. In particular embodiments, the biocompatible and hemocompatible polymer may be a polymer coated with a bioactive coating or surface-modified to include a bioactive material, and in such embodiments, the bioactive material may be an anti-coagulant coating, coumadin, heparin, a heparin derivative, a Factor Xa inhibitor, a direct thrombin inhibitor, hementin, sintered porous titanium microspheres, carbon coating, and combinations thereof. In some embodiments, each leaflet may have a ratio of width of the leaflet to a portion of a circumference of the conduit between fixing points of about 0.63 to about 1, and in certain embodiments, each leaflet may have a ratio of width of the leaflet to conduit diameter of about 0.9 to about 1.7. In some embodiments, the valve may further include a stent attached to an outer surface of the conduit.

Other embodiments are directed to stents including: a stent having an inner stent surface and an outer stent surface; and a valve structure comprising one or more leaflet, each leaflet having an outer sinus edge, an inner sinus edge, an open sinus edge and a fan, attached to the inner surface of the conduit at the outer sinus edge and inner sinus edge, wherein the open sinus edge is suspended below the inner surface of the conduit by a depth creating a sinus between the leaflet and the inner conduit surface. In some embodiments, each leaflet has a substantially triangular shape. In certain embodiments, each open sinus edge has a width that is less than the circumference of the conduit between fixture points of the leaflet to the conduit, and in particular embodiments, the outer sinus edge and the inner sinus edge may be attached to the conduit by a fluid impervious connection such as, but not limited to, suturing, welding, fusion, applying an adhesive, and combinations thereof. In various embodiments, the conduit and valve structure may each individually be composed of any biocompatible and hemocompatible polymer, and in such embodiments, the biocompatible and hemocompatible polymer may be a fluoropolymer such as, but not limited to, polytetrafluoroethylene, expanded polytetrafluoroethelyne, polyester, polyethylene terephthalate, polydimethylsiloxane, polyurethane, and combinations thereof. In particular embodiments, the biocompatible and hemocompatible polymer may be a polymer coated with a bioactive coating or surface-modified to include a bioactive material, and in such embodiments, the bioactive material may be an anti-coagulant coating, coumadin, heparin, a heparin derivative, a Factor Xa inhibitor, a direct thrombin inhibitor, hementin, sintered porous titanium microspheres, carbon coating, and combinations thereof. In some embodiments, each leaflet may have a ratio of width of the leaflet to a portion of a circumference of the conduit between fixing points of about 0.63 to about 1, and in certain embodiments, each leaflet may have a ratio of width of the leaflet to conduit diameter of about 0.9 to about 1.7. In some embodiments, the stent may further include conduit disposed between the stent and the valve.

Further embodiments, are directed to methods for making a valve including the steps of: inverting a conduit having an inner surface and an outer surface such that the inner surface is outward facing; bending a portion of the conduit along a longitudinal axis to create a tapered dimple; attaching a valve structure comprising one or more leaflet, each leaflet having an outer sinus edge, an inner sinus edge, an open sinus edge and a fan, by attaching the outer sinus edge and the inner sinus edge to the inner surface of the conduit at the tapered dimple; and reverting the conduit, thereby producing a valve structure on the inner surface of the conduit wherein the open sinus edge is suspended below the inner surface of the conduit by a depth creating a sinus between the leaflet and the inner conduit surface. In some embodiments, bending can be carried out using a fixture stencil. In certain embodiments, bending may further include mechanically deforming the conduit, heating the conduit, vacuum deforming the conduit, or combinations thereof.

Yet other embodiments include fixing stencils including: a handle; a stencil head attached at an end of the handle, the stencil head having a triangular flat surface having a base, a first outer edge, a second outer edge, and a tip, and a triangular curved surface opposite the triangular flat surface, the triangular curved surface being tapered laterally from a longitudinal axis extending from the handle to first outer edge and from the longitudinal axis to the second outer edge and being curved from the base to the tip creating a substantially tetrahedral shape stencil head extending away from the handle. In some embodiments, the fixing stencil may further include holes or slots along the first outer edge and the second outer edge, in other embodiments, the stencil head may be pierceable or locally destructible along the first outer edge and second outer edge.

### BRIEF DESCRIPTION OF THE DRAWINGS

FIG. 1 shows a valved conduit in closed position, FIG. 1A, and open position, FIG. 1B.

FIG. 2 shows a valved conduit in open position, FIG. 2A, and closed position, FIG. 2B.

FIG. 3A is a schematic showing the position of a valve in relation to the conduit.

FIG. 3B shows a valved conduit in closed position with the lengths illustrated in FIG. 3A superimposed over the valve components.

FIG. 4 is an illustration of a valve leaflet structure.

FIG. 5A shows a valved conduit that has been inverted such that the valve structures are on an outward facing side of the conduit.

FIG. 5B shows a valved conduit with the valve structure on an inner surface of the conduit.

FIG. 6 is a schematic illustrating the tapered dimple created between the leaflet and an inner surface of the conduit in a longitudinal view, FIG. 6A, and a cross-sectional view, FIG. 6B.

FIG. 7A-D are schematic illustrations of the tapered dimple from various views.

FIGS. 8A and B show a fixing stencil.

FIG. 9 shows a stent in open position FIG. 9A and closed position FIG. 9B.

### DETAILED DESCRIPTION

Before the invention is described, it is to be understood that this invention is not limited to the particular systems, methodologies or protocols described, as these may vary. It is also to be understood that the terminology used herein is for the purpose of describing particular embodiments only, and is not intended to limit the scope of the present disclosure.

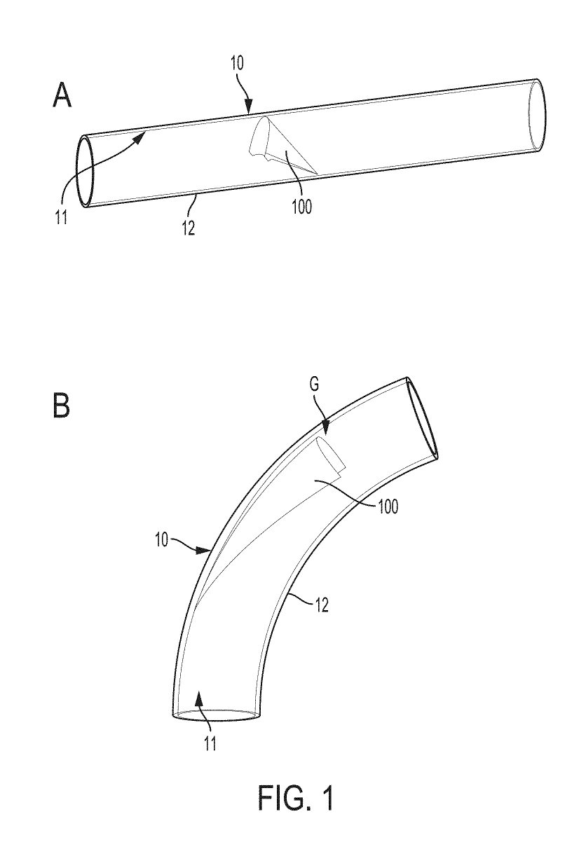

Various embodiments are directed to valved conduits having leaflets that do not contact the wall of the conduit in open position (FIG. 1B). As illustrated in FIGS. 1A and B, an example valved conduit encompassed by such embodiments may include a conduit 10 having an inner surface 11 and an outer surface 12. A valve 100 composed of one or more leaflets may be disposed within the conduit 10 and attached to the inner surface 11 of the conduit 10. In open position (FIG. 1B), a valve gap G separates the inner surface of the conduit 11 from the valve 100.

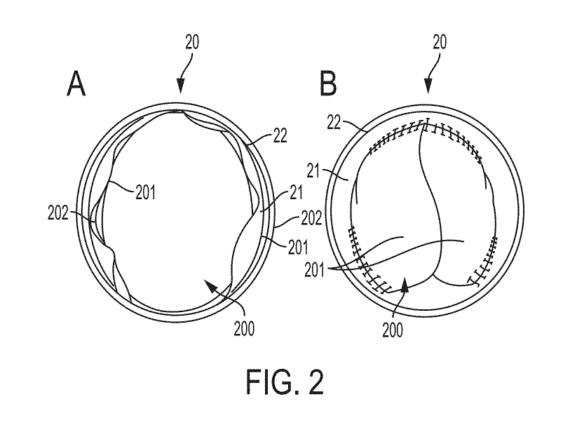

FIG. 2 is a cross-sectional view, showing a valve 200 in open FIG. 2A and closed FIG. 2B position. FIG. 2 illustrates an interior downstream view of an example valve encompassed by FIG. 1 in an open, FIG. 2A, and closed, FIG. 2B, configuration. In the open FIG. 2A configuration, fluid flows through the valve structure, forcing the fan portion of the leaflet 201 towards the inner surface of the conduit. In the closed configuration FIG. 2B the fan portion of the leaflet 201 may form a closure against fluid backflow. FIG. 2 shows a conduit 20 having an inner surface 21 and an outer surface 22. A valve 200 composed of one or more leaflets 201 that are attached to the inner surface 21 of the conduit 20. In open configuration (FIG. 2A), a gap area 202 is created between the leaflets 201 and the inner surface 21 of the conduit 20 that allows the leaflets 201 to fully extend without contacting the inner surface 21 of the conduit 20. In embodiments such as those depicted in FIG. 2 in which the valve includes two leaflets, at least a portion of the leaflets 201 may overlap along a diameter of the conduit 20 when in closed configuration (FIG. 2B), thereby substantially blocking flow of fluid through the conduit 20. In embodiments in which the valve includes one leaflet (not depicted), the leaflet may contact the inner surface of the conduit on an inner surface of the conduit opposite the attachment site of the valve, and in embodiments in which the valve includes three or more leaflets (not depicted), the leaflets may overlap at a longitudinal axis of the tube.

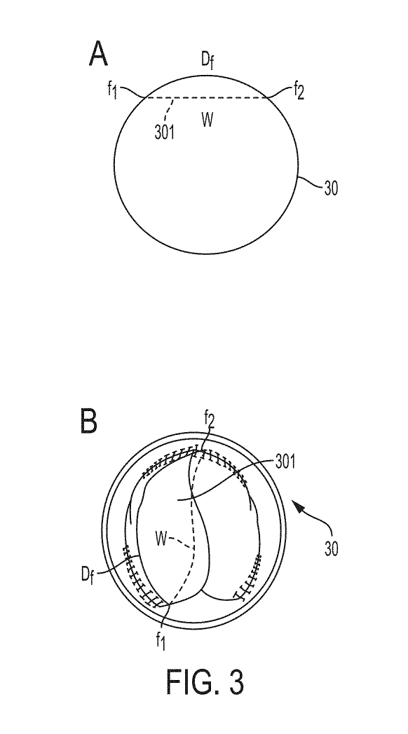

The gap area 202 between the inner surface 21 of the conduit 20 and the leaflet can be created by any means. For example, in some embodiments, the width W of the leaflets 201 may be shorter than the length of conduit between fixture points, D_{f}. This arrangement is illustrated in FIG. 3. FIG. 3A shows a simple diagram of the valve configuration in which the leaflet 301 of a valve encompassed by the embodiments described above is disposed within a conduit 30 such that the width, W (dashed line), of the leaflet 301 is shorter than the portion of the conduit, D_{f}, between a first fixture point, f₁, connecting the leaflet 301 to the inner surface 31 of the conduit 30 and a second fixture point, f₂, connecting the leaflet to the inner surface 31 of the conduit 30. This valve configuration is further illustrated in FIG. 3B using the example valve depicted in FIG. 2B. The portion of the conduit, D_{f}, between the first fixture point, f₁, and the second fixture point, f₂, is longer than the width, W (dashed line), of the leaflet 301.

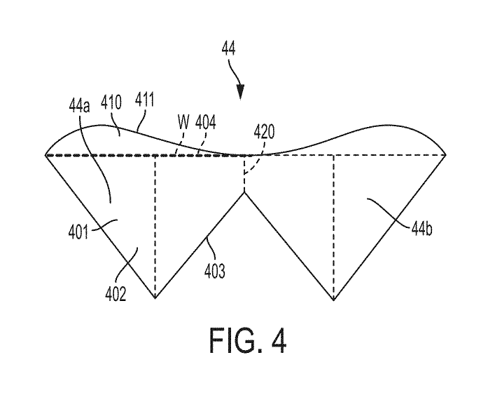

FIG. 4 is an illustration of the leaflet structure 44 unfolded on single plane with the width, W, of the leaflet illustrated in FIG. 2B identified (dashed line). In some embodiments, the leaflet structure may have additional features illustrated in FIG. 4. Although FIG. 4 shows a leaflet structure 44 configured to create a two leaflet valve, having a first leaflet 44a and a second leaflet 44, a leaflet structure for a single leaflet valve or leaflet structure for a three or four leaflet valve may include the same elements in a similar configuration.

Each leaflet 401a, 401b of such leaflet structures may include an outer sinus edge 402a, 402b, an inner sinus edge 403a, 403b, and an open sinus edge 404a, 404b. In embodiments in which the leaflet structure includes two or more leaflets, the open sinus edge 404a, 404b of each leaflet 401a, 401b may by coextensive as illustrated in FIG. 4. In some embodiments, the leaflet structure 44 may have a commissure 420 connecting the first leaflet 401a and the second leaflet 401b. In particular embodiments, the commissure 420 may be a perpendicular intersection connecting each inner sinus edge 403a, 403b with the meeting point of the open sinus edges 404a, 404b, creating a linear connection perpendicular to the open sinus edges 404a, 404b and at an angle to the inner sinus edges 403a, 403b.

In some embodiments, the leaflet structure 44 may further include a fan 410, having a fan edge 411, extending beyond the open sinus edge 404 away from the outer sinus edge 401 and inner sinus edge 402. The fan 410 may allow the leaflets of the valve to contact one another or overlap when the valve is in the closed position (see FIG. 3B) stopping flow of fluid through the valve. The fan 410 may have any shape, and in certain embodiments, the fan 410 may have a curved shape with a wide section on one side of the leaflet valve 44a and a narrow section on the opposite side of the leaflet valve 44a. In some embodiments, the narrow section of the fan 410 of a first leaflet 401a may connect to a narrow section of the second leaflet 401b at the commissure 420, and in particular embodiments, the narrow section of the fan 410 of a first leaflet 401a may connect to a narrow section of the second leaflet 401b at the commissure 420 at the connection point of the open sinus edges 404a, 404b.

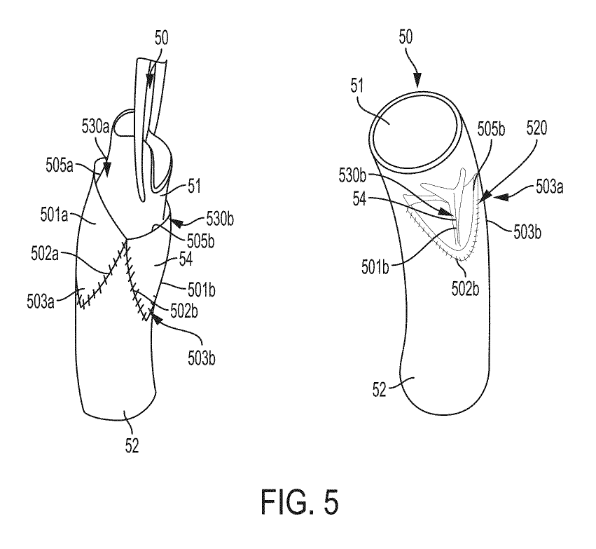

FIG. 5 shows a valve leaflet structure 54 such as that described in FIG. 4 attached to a conduit 50. In FIG. 5A, the conduit 50 is inverted such that the valve leaflet structure 54 is disposed on the outside of the conduit 50, and the valve leaflet structure is reverted such that the valve leaflet structure 54 is inside the conduit 50. Thus, the inner surface 51 is on the outside of the conduit 50 in FIG. 5A, and the inner surface 51 is inside the conduit 50 in FIG. 5B. The outer surface 52 is on the inside of the conduit 50 in FIG. 5A, and the outer surface 52 is outside the conduit 50 in FIG. 5B. The valve leaflet structure 54 may be attached to the inner surface 51 of the conduit 50 at the outer sinus edge 502a and 502a and the inner sinus edge 503b (503a is on the opposite side of the conduit 54). Each of the outer sinus edges 502a, 502b and inner sinus edge 503a, 503b may be attached to the conduit by a substantially fluid impervious connection such as, for example, suturing (as shown), fusion, applying an adhesive, or welding. The commissure 520 may also be attached to the inner surface 51 of the conduit 50 by, for example, suturing (as shown), applying an adhesive, or welding. The open sinus edge 504a, 505b are not attached to the conduit 50, and remain open to fluids flowing through the conduit 54. The opening creates a sinus 530a, 530b between the inner surface 51 of the conduit 50 and each leaflet 501a, 501b, and each open sinus edge 505a, 505b.

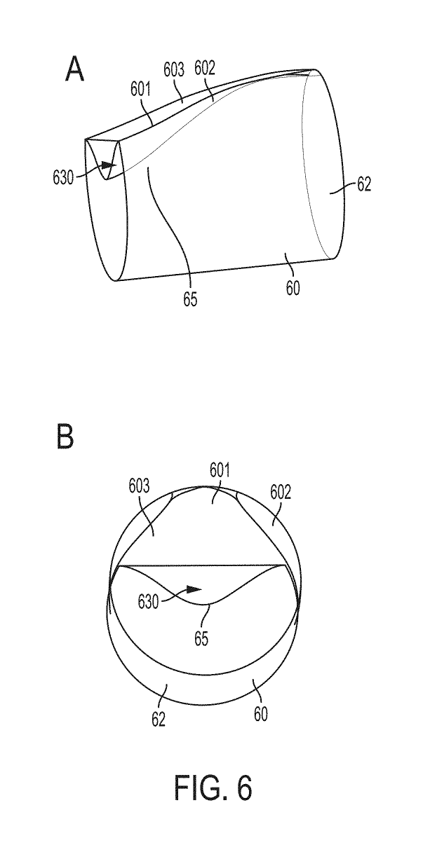

FIG. 6 is a three-dimensional representation of a single leaflet 601 (dark shading) on an inverted conduit 60 to show the sinus 630 created by the leaflet 601. FIG. 6A is a longitudinal view and FIG. 6B is a cross-sectional view. As in FIG. 5A, the leaflet 601 is attached to the conduit at the outer sinus edge 602 and inner sinus edge 603 by a substantially fluid impervious connection such as, for example, suturing, applying an adhesive, or welding. A tapered dimple 65 in the conduit 60 underlying the leaflet 601 provides the conduit side of the sinus 630 and allows the valve to achieve the configuration illustrated in FIG. 3A when the conduit 60 is returned to its original shape (i.e. reverted such that the outer surface 62 of the conduit 60 is on an outer surface of the structure). Because the width of the leaflet 601 changes along its length, the degree to which the conduit must bend also changes along the length of conduit 60.

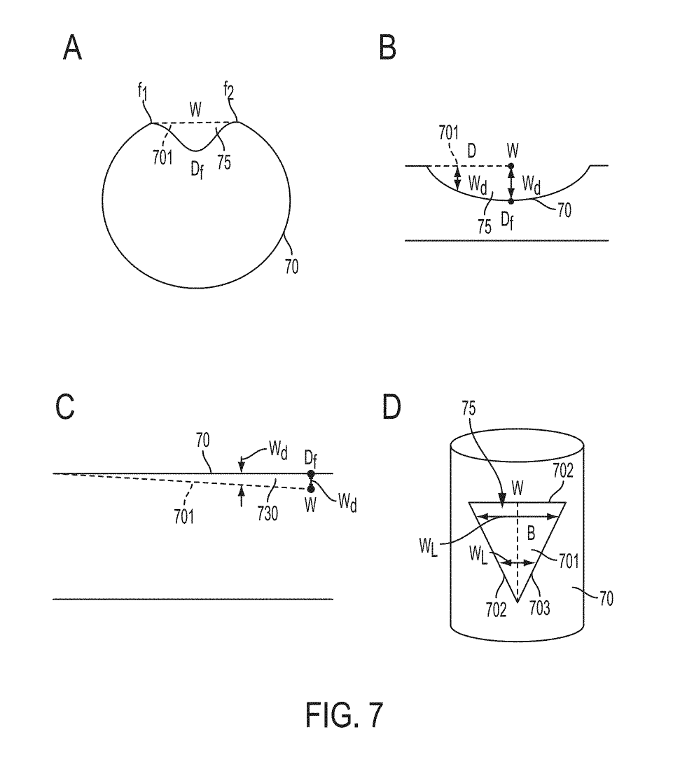

This arrangement is further illustrated in FIG. 7. In FIG. 7A, the valve is in an inverted configuration and shows the leaflet 701 of a valve disposed within a conduit 70 such that the width, W (dashed line), of the leaflet 701 is shorter than the portion of the conduit, D_{f}, between a first fixture point, f₁ and a second fixture point, f₂, connecting the leaflet to the inner surface of the conduit 70. FIG. 7B is a perpendicular cross section of the valve structure of FIG. 7A illustrating the tapered dimple 75. B is the length of the leaflet 701. W_{d} depth of the dimple 75, i.e. the depth of the gap between the leaflet 701 and the conduit 70, which, as illustrated varies with B from the open edge 704 of the leaflet 701 to a point where the outer sinus edge 702 and the inner sinus edge 703 meet (see FIG. 7D). FIG. 7C and FIG. 7D show the valve in operable configuration where the conduit has been reverted such that the valve is on the inner surface of the conduit 70 and conduit 70 has retained its cylindrical shape. The leaflet 701, which has an open edge 704 width, W, that is less than the circumference of the conduit 70 between fixture points may be suspended below the inner surface of the conduit 70 by a depth, W_{d}, which varies with the length, B, of the leaflet 701 creating a sinus 730. With reference to FIG. 7D, in some embodiments, the leaflet 701 may have a substantially triangular shape. Therefore, the leaflet width, W_{L}, may also vary with the length, B, of the leaflet 701.

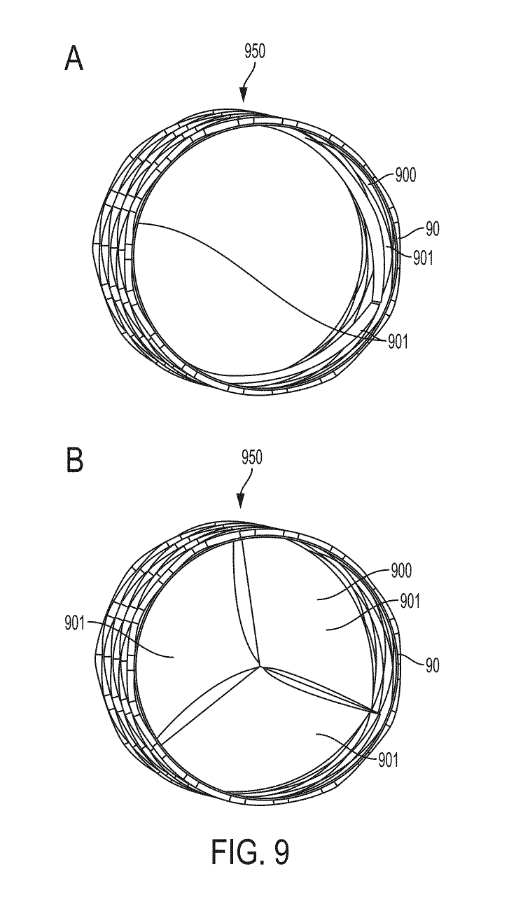

Some embodiments are directed to a stent 950 containing a valve 900 such as those described above. Although FIG. 9 shows a stent 950 having a conduit 90 disposed on an inner surface of the stent 950, in some embodiments, the valve may be attached directly to the inner surface of the stent 950, without conduit 90 disposed between the valve 900 and the stent 950. In another embodiment, a conduit may also be located on the outer surface of the stent. A conduit on the outer, inner, or both surfaces of the stent may cover all or only a portion of the length and circumference of the stent. The valves contained within the stents of such embodiments can have any number of leaflets 901. For example, the stent 950 in FIG. 9 has three leaflets 901; however, embodiments include stents 950 with valves 900 having two leaflets as illustrated in FIG. 2 and FIG. 3 or one leaflet as illustrated in FIG. 1. Similarly, the valved conduits described above and exemplified in FIG. 1-3 can include three leaflets as illustrated in FIG. 9. Each leaflet 901 of the stent 950 of various embodiments may include a valve gap (not depicted) when the valve is in open position (FIG. 9A) and therefore, may have a width that is shorter than the portion of the conduit between fixture points. The stent 950 of such embodiments can be made from any material known in the art such as, for example, wire mesh, and the mesh can be in any configuration. In some embodiments, the stent may be a commercially available stent into which a valve or valved conduit has been inserted and attached.

A valve having leaflets as described and discussed above may reduce the contact of the leaflets and, in some embodiments, fans attached to the open edge, with the inner surface of the conduit when the valve is in open configuration. Reduced contact with the inner surface of the conduit decreases the likelihood that the valve will stick in open configuration and may also reduce wear on the leaflet over many cycles. Thus, the valves of various embodiments may provide improved long term use when implanted as part of a medical device. For example, in some embodiments, the valves described above may be used as a shunt for connecting of the right ventricle to the pulmonary artery following a Norwood operation, as frequently performed for the treatment of single-functional-ventricle-disorders such as Hypoplastic Left Heart Syndrome. In other embodiments, the valves described above may be used for the correction or reconstruction of the right ventricle outflow tract (RVOT) for congenital heart disorders such as tetralogy of Fallot, Truncus Arterious, DextroTransposition of the Great Arteries, Pulmonary Atresia of Intact Ventricular Septum, or Aortic Valvular Disease. In still other embodiments, the valves described above may be incorporated into a stent and deployed as artificial valves in adult and pediatric patients.

The conduit 10, 20, 30, 40, 50, 60, 70 and stents 90 of various embodiments, and the valve structure 100, 200, 300, 400, 500, 600, 700, 900 may be composed of any biocompatible and hemocompatible polymer. For example, in some embodiments, biocompatible and hemocompatible polymer may be a fluoropolymer, and in certain embodiments, the biocompatible and hemocompatible polymer may be polytetrafluoroethylene, expanded polytetrafluoroethelyne, polyester, polyethylene terephthalate, polydimethylsiloxane, polyurethane, and combinations thereof. In some embodiments, the biocompatible and hemocompatible polymer may be a polymer coated with a bioactive coating, or in other embodiments, the biocompatible and hemocompatible polymer may be surface-modified to include a bioactive material. The bioactive coating or bioactive material may be an anti-coagulant coating or material that promotes biocompatibility such as, for example, coumadin, heparin, a heparin derivative, a Factor Xa inhibitor, a direct thrombin inhibitor, hementin, sintered porous titanium microspheres, a carbon coating, or combinations thereof.

The conduit 10, 20, 30, 40, 50, 60, 70, 90 may generally be flexible, and the size of the conduit of various embodiments may vary depending on the intended use of the valve. For example, in some embodiments, the conduit 10, 20, 30, 40, 50, 60, 70, 90 may have a diameter to of about 40 mm to about 15 mm, 25 mm to about 2 mm, about 20 mm to about 2 mm, about 15 mm to about 2 mm, about 10 mm to about 2 mm, about 8 mm to about 3 mm, about 5 mm to about 3 mm, or any range or individual diameter encompassed by these example ranges. In other embodiments, the conduit 10, 20, 30, 40, 50, 60, 70, 90 may have a diameter of about 40 mm to about 15 mm, 25 mm to about 5 mm, about 20 mm to about 8 mm, about 15 mm to about 10 mm, or any range or individual diameter encompassed by these example ranges. The conduit 10, 20, 30, 40, 50, 60, 70, 90 may have a various thicknesses depending on the intended use. For example, in various embodiments, the conduit may have a thickness of about 0.05 mm to 0.5 mm, 0.5 mm to about 2.0 mm, about 0.5 mm to about 1.5 mm, or any range or individual thickness encompassed by these example ranges.

The valve structure 100, 200, 300, 400, 500, 600, 700, 900 may have a thickness of about 0.05 mm to about 0.3 mm in various embodiments, and this thickness may vary within the valve structure. For example, in some embodiments, the leaflet may have a greater thickness than a fan or a fan may have a greater thickness than the leaflet. The thickness of such valve structures may be selected to provide sufficient flexibility to allow the valve to obtain the open and closed configurations under the pressure of the flow of fluid through the conduit. The dimensions of each leaflet 101, 201, 301, 401, 501, 601, 701, 901 of the valve structure 100, 200, 300, 400, 500, 600, 700, 901 may vary depending on the diameter of the conduit 10, 20, 30, 40, 50, 60, 70, 90 and the number of leaflets 101, 201, 301, 401, 501, 601, 701 making up the valve structure 100, 200, 300, 400, 500, 600, 700, 901. For example with reference to FIG. 7D, in various embodiments, ratio the length, B, of a leaflet 701, and the width, W, leaflet may be about 0.2 to about 2, about 0.3 to about 2, about 0.4 to about 2, about 0.5 to about 2, about 0.6 to about 2, about 0.75 to about 2, about 1 to about 2, about 1 to about 1, or any ratio therebetween or any ratio encompassed by these example ratios. In some embodiments, the ratio of the width of the leaflet to a portion of the conduit circumference between the fixing points may be about 0.63 to about 1, about 0.7 to about 1, about 0.5 to about 1, or any ratio therebetween or any ratio encompassed by these example ratios. The ratio of the width, W, the leaflet 701 to the diameter of the conduit may be about 0.02 to about 3, about 0.05 to about 3, about 0.08 to about 3, about 0.1 to about 3, about 0.2 to about 3, about 0.5 to about 3, about 1 to about 3, about 0.9 to about 1.7, or any ratio therebetween or any ratio encompassed by these example ratios. In embodiments, including a commissure 420 (FIG. 4) i.e. valves having more than one leaflet, the ratio between a length of the commissure 420 and the width, W, of the leaflet 401, may be about 0.05 to about 2, about 0.1 to about 2, about 0.2 to about 2, about 0.3 to about 2, about 0.5 to about 2, or any ratio therebetween or any ratio encompassed by these example ratios. The ratio of the inner sinus edge 703 the leaflet 701 to the width, W, of the leaflet 701 may be about 0.2 to about 2.5, about 0.3 to about 2.5, about 0.4 to about 2.5, about 0.5 to about 2.5, about 0.6 to about 2.5, about 0.75 to about 2.5, about 1 to about 2.5, about 1 to about 1, or any ratio therebetween or any ratio encompassed by these example ratios.

In various such embodiments, the width, W, of the leaflet 701 may be about 1 mm to about 10 mm, about 2 mm to about 7 mm, about 2 mm to about 5 mm, about 20 mm to about 40 mm, about 10 mm to about 30 mm, or any individual width or range encompassed by these example widths. The length, B, of the leaflet 701 may be about 5 mm to about 40 mm, about 5 mm to about 30 mm, about 8 mm to about 25 mm, about 10 mm to about 20 mm, or any individual length, B, or range encompassed by these example lengths. The length of the inner sinus edge 703 about outer sinus edge 704 may each, individually, be about 5 mm to about 45 mm, about 5 mm to about 35 mm, about 8 mm to about 30 mm, about 10 mm to about 20 mm, or any individual length or range encompassed by these example lengths. In some embodiments, multiple leaflet valves may have no commissure, and in other embodiments, multiple leaflet valves may have a commissure having a length of about 0.05 mm, about 0.1 mm, about 0.2 mm, about 0.3 mm, about 0.5 mm, about 0.8 mm, about 1.0 mm, about 1.5 mm, about 2.0 mm, about 4.0 mm, about 6.0 mm, about 8.0 mm, about 10.0 mm, about 12.0 mm, or any individual length encompassed by these example lengths.

Although FIGS. 2-7 illustrate leaflet structures composed of one or two leaflets, the valve structure of other embodiments may be composed of any number of leaflets. For example, embodiments include a valve structure having three and four leaflets in which each leaflet has an inner and outer sinus edge, a fan edge, a fan, and a commissure between each neighboring leaflet. For example, a three-leaflet valve structure may include, two commissures: one commissure between a first leaflet and a second leaflet, and a second commissure between the second leaflet and a third leaflet. Equivalent metrics to those described above can be used to describe each leaflet of a multi-leaflet valve structure. The valve structure may incorporate a closure formed by the juxtaposition, proximity, and/or overlap of three or four fan structures. The mutual disposition of some portions of the three or four fan edges along with the inner surface of the conduit may result in a gap similar to the gap area described above.

The valves 100, 200, 300, 400, 500, 600, 700, 900 described above are not limited to a particular utility. For example, in some embodiments, the valves described above 100, 200, 300, 400, 500, 600, 700, 900 can be used as heart valves 1 for use in cardiac, coronary or vascular procedures, which may be composed of one or more heart valve leaflets. The term may encompass, as non-limiting examples, a heart valve single leaflet structure having a single heart valve leaflet, or a heart valve multi-leaflet structure having more than one heart valve leaflet. Each heart valve leaflet may include a sinus edge, a fan edge, a sinus structure, and a fan structure, and additional structural components such as, without limitation, a conduit (which may be tube-like, stent-like, or multi-layered such as a tube within a stent) and one or more conduit sinus structures. The term may encompass a single leaflet valve structure having a valve single leaflet structure, or a multi-leaflet valve structure composed of either multiple valve single leaflet structures or a valve multi-leaflet structure.

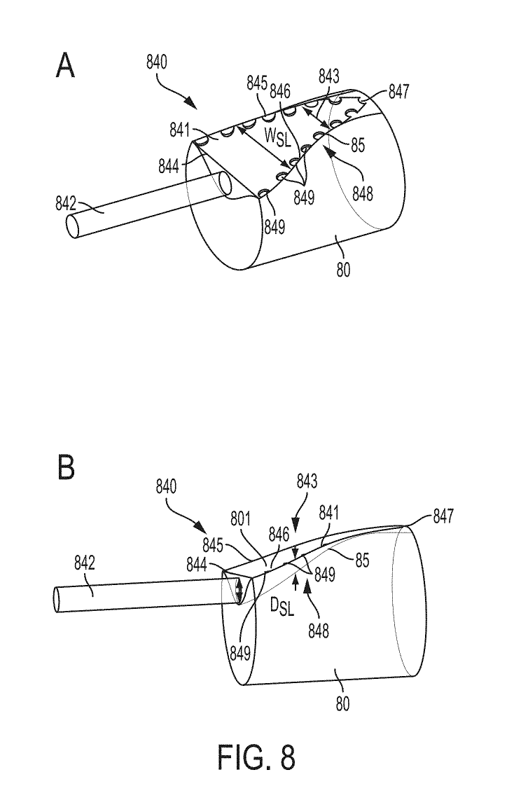

Various embodiments are directed to a fixing stencil 840. In some embodiments as illustrated in FIG. 8, the fixing stencil 840 may include a stencil head 841 and handle 842. The stencil head 841 may substantially triangular flat surface 843 having a base 844, a first outer edge 845, a second outer edge 846, and a tip 847, and a triangular curved surface 848. In various embodiments, the triangular curved surface 848 may be tapered laterally from a longitudinal axis extending from the handle 842 to first outer edge 845 and from the longitudinal axis to the second outer edge 847. The triangular curved surface 848 may be further curved from the base 844 to the tip 847 creating a complex of curved surfaces having a tetrahedral shape extending away from the base 844 to the tip 847. In some embodiments, the may further include holes or slots 849 along the first outer edge 845 and second outer edge 846 to allow physical fixturing, for example, suturing, welding, adhesive application, or other means for attaching the leaflet 801 to the conduit 80 without contacting the stencil head 841. In other embodiments, the stencil head 841 may be pierceable or locally destructible along the first outer edge 845 and second outer edge 846 to allow removal of the stencil head 841 after fixturing.

The handle 842 of various embodiments may be any means for manipulating and holding the fixing stencil 840 in place while the valve is attached to the conduit. For example, in some embodiments, the handle may be sized and shaped to be gripped by a human hand. In other embodiments, the handle may be include one or more tabs or wings sized and shaped for holding the fixing stencil to the conduit and valve using surgical tools, claps, vice grips, or other tools.

The size of the stencil head 841 may vary depending on the size and shape of the valve to 8 that will be produced using the fixing stencil 840. In general, the length, Bₛ, from the base 844, extending from the handle, to the tip 845, of the stencil head 841 may be substantially the same length, B, as the leaflet 801. The variable width, W_{SL}, of the substantially triangular flat surface may substantially correspond to the variable width of the triangular leaflet and the variable depth, D_{SL}, of the triangular curved surface 884 may substantially corresponding to the depth of the sinus 830. Thus, the stencil head may be configured to have substantially the same shape and volume as the sinus 830 created between the leaflet 801 and the portion of the conduit 80 making up the tapered dimple 85.

In various embodiments, the width, W_{SL}, of the stencil head 841 may be about 1 mm to about 10 mm, about 2 mm to about 7 mm, about 2 mm to about 5 mm, about 20 mm to about 40 mm, about 10 mm to about 30 mm, or any individual width or range encompassed by these example widths. The length, B_{S}, of the stencil head 841 may be about 5 mm to about 40 mm, about 5 mm to about 30 mm, about 8 mm to about 25 mm, about 10 mm to about 20 mm, or any individual length or range encompassed by these example lengths. The length of the first outer edge 845 and second outer edge 846 may each, individually, be about 5 mm to about 45 mm, about 5 mm to about 35 mm, about 8 mm to about 30 mm, about 10 mm to about 20 mm, or any individual length or range encompassed by these example lengths. The depth, D_{SL}, of the stencil head 841 at the base 844 may be about 1 mm to about 10 mm, about 1 mm to about 7 mm, about 1 mm to about 5 mm, or any individual depth or range encompassed by these example depths.

In some embodiments, the fixing stencil 840 may further include stabilizing components the stencil head 841 during fixturing. For example, the stabilizing components may include a clamp positioned to hold the tapered dimple 85 or an apparatus that substantially fills the remainder of the conduit. In certain embodiments, the stencil head 841 may be capable of transmitting heat to the conduit to aid in fixturing by fusing the leaflet 801 to the conduit 80 or aiding in deformation of the conduit 80 creating a sinus bulge at the sinus when the fixing stencil 840 is removed.

Further embodiments are directed to methods for making the valved conduits described above. Such embodiments may include the steps of inverting a conduit, bending a portion of the conduit to create a tapered dimple, attaching a leaflet to the conduit on a surface surrounding the tapered dimple, and reverting the conduit placing the leaflet on an inner surface of the conduit. The step of attaching can be carried out by suturing, welding, fusing, using an adhesive, and the like or combinations thereof. In some embodiments, the method may further include the step of deforming the conduit to produce a sinus bulge. In some embodiments, bending can be carried out using a fixing stencil configured to hold conduit in a bent form creating a tapered dimple. The fixing stencil may have substantially the same shape as the tapered dimple. In various embodiments, the fixing stencil may have one or more of the parts described above.

In some embodiments, the method may include the step of cutting a valve structure from a biocompatible material. In certain embodiments, the step of cutting the valve structure may be preceded by a step of marking the biocompatible material, and in some embodiments, marking may be carried out by tracing a valve structure stencil having substantially the same shape as the valve structure onto the biocompatible material. In other embodiments, the marking can be carried out using a stamp having substantially the same shape as the valve structure. In still other embodiments, step of cutting can be carried out using a die cutting machine.

In some embodiment, the method may further include the step of marking an inner surface of the conduit with a location form attaching the leaflet to the conduit, thereby providing proper placement and alignment of the leaflets. Marking can be carried out by various means. For example, in some embodiments, marking can be carried out using a sinus stencil may be provided, and the marking on the inner surface of the conduit can be substantially the same as the sinus stencil. In such embodiments, the sinus stencil may have a shape and dimension for showing the location of the tapered dimple on the unbent inner surface of the conduit. Therefore, the sinus stencil may be wider than the valve structure, but the markings may have essentially the same shape as the leaflet after the conduit is bent.

1-26. (canceled)

27. A valve comprising:
a conduit having an inner conduit surface and an outer conduit surface; and
a valve structure comprising one or more leaflet, the one or more leaflet having an outer sinus edge, an inner sinus edge, an open sinus edge and a fan, wherein the valve structure is attached to the inner conduit surface at the outer sinus edge and inner sinus edge, wherein the open sinus edge is suspended below the inner conduit surface creating a sinus between the leaflet and the inner conduit surface.

28. The valve of claim 27, wherein the leaflet has a substantially triangular shape.

29. The valve of claim 27, wherein the open sinus edge has a width that is less than the circumference of the conduit between fixture points of the leaflet to the conduit.

30. The valve of claim 27, wherein the outer sinus edge and the inner sinus edge are attached to the conduit by a fluid impervious connection selected from the group consisting of suturing, welding, fusion, applying an adhesive, and combinations thereof.

31. The valve of claim 27, wherein the conduit and the valve structure are each individually composed of a biocompatible and hemocompatible polymer.

32. The valve of claim 31, wherein the biocompatible and hemocompatible polymer is a fluoropolymer selected from the group consisting of polytetrafluoroethylene, expanded polytetrafluoroethelyne, polyester, polyethylene terephthalate, polydimethylsiloxane, polyurethane, and combinations thereof.

33. The valve of claim 31, wherein the biocompatible and hemocompatible polymer is a polymer coated with a bioactive coating or surface-modified to include a bioactive material.

34. The valve of claim 33, wherein the bioactive material is selected from the group consisting of anti-coagulant coatings, coumadin, heparin, a heparin derivative, a Factor Xa inhibitor, a direct thrombin inhibitor, hementin, sintered porous titanium microspheres, carbon coating, and combinations thereof.

35. The valve of claim 27, wherein the leaflet has a ratio of width of the leaflet to a portion of a circumference of the conduit between fixing points of about 0.63 to about 1.

36. The valve of claim 27, wherein the leaflet has a ratio of width of the leaflet to conduit diameter of about 0.9 to about 1.7.

37. The valve of claim 27, wherein the conduit comprises a stent having an inner stent surface and an outer stent surface.

38. A method for making a valve comprising:
inverting a conduit having an inner surface and an outer surface such that the inner surface is outward facing;
bending a portion of the conduit along a longitudinal axis to create a tapered dimple;
attaching an outer sinus edge and an inner sinus edge of a valve structure to the inner surface of the conduit at the tapered dimple, wherein the valve structure comprises one or more leaflet, the one or more leaflet having the outer sinus edge, the inner sinus edge, an open sinus edge, and a fan; and
reverting the conduit, thereby producing the valve structure on the inner surface of the conduit wherein the open sinus edge is suspended below the inner surface of the conduit by a depth creating a sinus between the one or more leaflet and the inner conduit surface.

39. The method of claim 38, wherein bending is carried out using a fixture stencil.

40. The method of claim 38, wherein bending further comprises mechanically deforming the conduit, heating the conduit, vacuum deforming the conduit, or combinations thereof.

41. A fixing stencil comprising:
a handle having an end;
a stencil head attached at the end of the handle, the stencil head having a triangular flat surface comprising a base, a first outer edge, a second outer edge, and a tip, and a triangular curved surface opposite the triangular flat surface, the triangular curved surface being tapered laterally from a longitudinal axis extending from the handle to first outer edge and from the longitudinal axis to the second outer edge and being curved from the base to the tip creating a substantially tetrahedral shape stencil head extending away from the handle.

42. The fixing stencil of claim 41, further comprising holes or slots along the first outer edge and the second outer edge.

43. The fixing stencil of claim 41, wherein the stencil head is pierceable or locally destructible along the first outer edge and second outer edge.
